(19) United States
(12) Patent Application Publication
(10) Pub. No.: US 2007/0292361 A1
(43) Pub. Date: Dec. 20, 2007

(54) Encapsulation compositions

(75) Inventors: Teresa Thomas Virgallito, Beavercreek, OH (US); Robert B. Wieland, Cincinnati, OH (US); Michael Chaney, Ludlow, KY (US)

Correspondence Address: CURATOLO SIDOTI CO., LPA, 24500 CENTER RIDGE ROAD, SUITE 280, CLEVELAND, OH, 44145, US

(73) Assignee: Givaudan S.A., Vernier (CH)

(21) Appl. No.: 11/811,819

(22) Filed: Jun. 12, 2007

Related U.S. Application Data
(60) Provisional application No. 60/813,317, filed on Jun. 13, 2006.

(51) Int. Cl. A61K 8/18; A61K 9/50; A61K 9/16; C11D 17/02

(52) U.S. Cl. 424/49; 424/401; 424/490; 510/295

(57) ABSTRACT

Microcapsules having hydrogel walls and a content, the content including a flavour or fragrance active and a solvent therefor, the solvent having a Clog P>5, the solvent being present in such a proportion that the active in solution has a calculated base-ten logarithm of the partition coefficient between the solvent and an continuous aqueous phase containing 1.5% by weight anionic surfactant of at least 1.7. The capsules are useful in providing actives in high surfactant compositions, such as toothpastes and tooth-gels, in which the proportion of active remaining in the capsules on storage is appreciably higher than that achievable by conventional encapsulation techniques.

### CROSS REFERENCE TO RELATED APPLICATIONS

This application claims the benefit of the filing date, under 35 USC §119(e), from U.S. Provisional Application No. 60/813,317 filed Jun. 13, 2006, which is incorporated by reference as if fully written out below.

### TECHNICAL FIELD

Disclosed are flavours and fragrances in encapsulated form, and encapsulated forms, particularly for use in high surfactant systems.

### BACKGROUND

The use of encapsulated flavours and fragrances (hereinafter “active” or “actives”) in products is well known and has been widely used in numerous applications. It is an excellent method of preserving actives until they are needed, at which point they are released by rupturing the capsules. This generally occurs when the product is subjected to conditions causing the rupturing. For example, in a toothpaste, the capsules may be ruptured by brushing teeth.

Until that point, the capsules should ideally maintain the active content with which they were initially loaded. This is generally possible in many cases, with only small (and acceptable) losses as a result of premature capsule rupture and active leaking through capsule walls. However, in some cases, the losses are unacceptably high. This occurs when a capsule that has a hydrogel shell, that is, a shell of crosslinked, water-swellable polymer, is exposed to a high surfactant continuous phase, such as that of a toothpaste.

The problem that occurs with the hydrated capsule walls of the hydrogel capsules in a high surfactant system or environment (about 1-10% by weight of the total composition) is that the active, especially if it is an oily material, can be leached out by the continuous phase. Unfortunately, some of the more desirable actives, such as peppermint oil for toothpastes and tooth gels, are this sort of material and suffer particularly from this problem. It has hitherto proved impossible to keep the losses of such actives in such an environment to acceptable levels.

### SUMMARY

It has now been found that it is possible to prepare hydrogel shell capsules that can retain most or even all of their active content, even when exposed to a high-surfactant continuous phase. Therefore, provided are microcapsules comprising hydrogel walls and a content comprising a flavour or fragrance active and a solvent therefor, the solvent having a Clog P>5, the solvent being present in such a proportion that the active in solution has a calculated base-ten logarithm of the partition coefficient between the solvent and a continuous aqueous phase containing 1.5% by weight anionic surfactant (Log Pocs) of at least 1.7.

### BRIEF DESCRIPTION OF THE DRAWINGS

FIG. 1 is a graph showing flavour intensity rating vs. brushing cycle for mint capsules in a fresh unflavored tooth-gel compared with mint capsules in an aged unflavored tooth-gel.

FIG. 2 is a graph showing flavour intensity rating vs. brushing cycle for QFP sensory testing of flavour intensity delivered during brushing cycle.

FIG. 3 is a graph showing capsule hardness testing which plots percent of capsules ruptured vs. time comparing two core diameter to wall thickness ratios at each of two particle size ranges.

### DESCRIPTION

ClogP, the calculated base-ten logarithm of the octanol-water partition coefficient, is a very well known parameter, especially in the fabric care industry. The ClogP figures used for the purposes of this invention are those found in the Scifinder™ system of the Chemical Abstracts™ Service. These are calculated using the commercially-available Advanced Chemistry Development (ACD/Labs) Software, V8.14.

The logarithm of the partition coefficient for the oil-continuous phase system (hereinafter Log Pocs) is the logarithm of the ratio of the concentrations of the active in the active/solvent phase to the continuous phase after partitioning is complete. The standard continuous phase for this is a 1.5% by weight solution of an anionic surfactant in water. Partitioning experiments were run to determine the partition coefficient of chemicals in surfactant solution. The logarithm of the chemical partition co-efficient in surfactant solution (Log P_{OCS}) was calculated, as follows:
Log P_{OCS}=Log(C_{O}/C_{CS})
where C_{O} and C_{CS} are the concentrations (in g/cm³) of the chemical in the oil and surfactant solution phase, respectively.

In an illustrative embodiment, the Log P_{OCS} may be greater than 2. A satisfactory Log P_{OCS} may be easily achieved by the skilled person using the ordinary skill of the art for any combination of oil and continuous phase.

The active may be any suitable flavour or fragrance whose use is desired. There are many such materials. Illustrative examples include (but are not restricted to) peppermint oil, menthol, beta damascone, menthone, alpha ionone, alpha irone, neryl acetate, d-limonene, decanal, octanal, menthyl acetate, menthyl saticylate, allyl cyclohexane propionate and allyl octanoate.

It is possible to use more than one such oil, either in a mixture or separately in different capsules. A mixture of oils should comply with the Log P_{OCS} requirements hereinabove described.

The solvents for use in the microcapsules are any solvents that can partially or completely dissolve the desired active, provided that the desired Log P_{OCS} can be achieved. The selection of a suitable solvent or solvents is well within the skill of the art. Illustrative examples of suitable solvents include, di- and triglycerides, migylol, soybean oil, olive oil, paraffin oil, palmitic acid. soybean oil flakes , soybean-cotton seed flakes, paraffin wax, carnauba wax and beeswax.

The hydrogel capsules may be selected from any such suitable capsules known to the art. The capsule material is typically (but not always) of gelatine, alginate, pectin and carrageenan. According to certain illustrative embodiments, the microcapsules may comprise a blend of gelatine and carboxymethylcellulose (CMC). Such capsules are generally prepared by a complex coacervation method well known to and widely used by the art. A typical such method is to disperse the active in the form of droplets in an aqueous solution or dispersion of a microcapsule-forming polymer. This polymer is then caused to deposit on the active droplets and to harden.

The thickness of the capsule walls are selected to provide the best compromise between processing and storage stability on the one hand, and release of the active in desired circumstances. The skilled person can readily determine and achieve a suitable wall thickness. Without limitation, the ratio of the diameter of the capsule to the capsule wall thickness, R, is at least about 10:1. In other embodiments, R may be at least about 12:1, at least about 16:1, or at least about 20:1.

Capsule sizes are not critical and the typical sizes encountered in the art are also useful in the working and use of the subject microcapsules, compositions and methods. Without intending limitation thereto, in certain embodiments capsule sizes may range from about 100 to about 2500 micrometres (μm), and in other embodiments, about 100 to about 2000 μm.

In addition to the ingredients hereinabove specified, the capsules and/or the active may contain any other standard ingredients known to the art for particular properties, added in art-recognised quantities. One example is use of filler in the capsule walls for reinforcing and/or for price reduction. Any such filler known to the art may be used, but typical examples include cellulosic materials, such as microcrystalline cellulose and mineral fillers, such as talc, clays and silica.

The capsule material may be coloured, and this may be achieved by the addition of any suitable oil-soluble dye. Coloured pigments may also be used, and these can also provide a filling/reinforcing effect, as hereinabove described.

The capsules may be incorporated into compositions in which their presence is desired by any conventional means, such as low shear mixing. Illustrative, non-limiting examples of such compositions include toothpastes and tooth-gels, laundry detergents, fabric softeners, hair care products, such as shampoos and conditioners, cosmetic and medicinal creams, personal cleansing products such as shower gels, body lotions and soaps. In addition to the capsules, these compositions may comprise all or any of the standard art-recognised ingredients known to be useful in such compositions, in art-recognised proportions. The nature of these known ingredients will vary depending on the nature of the composition, but typical, non-limiting examples include pigments (colorants), fillers and extenders, thickening agents, rheology modifiers, fragrance/flavour additives, surfactants, preservatives and fixatives.

The capsules are especially useful in compositions with a high surfactant content, that is, those having a surfactant content between about 1 to about 10% by weight or higher. These provide especially difficult environments for long-term active retention, and conventional microcapsules will typically lose up to 90% and even as much as 100% encapsulated active on storage. However, the capsules (i.e., microcapsules) as hereinabove described retain more active in these harsh conditions.

Therefore, also provided is a composition having a surfactant content of from about 1 to about 10% by weight, the composition comprising encapsulated flavour or fragrance active provided in capsules as hereinabove described.

Additionally provided is a method of increasing the storage life of encapsulated flavour or fragrance active in a composition that constitutes a high surfactant environment, comprising incorporating the active in the microcapsules as hereinabove described and blending the microcapsules into the composition.

There now follows a series of non-limiting examples that serve to further illustrate the microcapsules, compositions and methods. The examples, which describe certain illustrative embodiments, should not be construed to limit the microcapsules, compositions or methods in any manner. Unless otherwise stated, all proportions are by weight.

### EXAMPLE 1

Hydrogel capsules were made using complex coacervation as the encapsulation process, using methods known to the art. Gelatin and CMC were the encapsulating materials. Two types of capsules were made. The first type had a core of a blend of 20 wt % citrus flavour, flavour blend having a calculated Log P_{OCS} of 2.3, and 80 wt % of dilution solvent migylol (MCT Oil). The second type had a core of 100 wt % citrus flavour. The capsules had a particle size range of 500 to 1000 microns. The flavored capsules were loaded into the following unflavored tooth-gel formulation, at 2 wt % load:

| | |
|---|---|
| Glycerol (98%) | 1.60 |
| thickener¹ | 0.30 |
| sorbitol (70%) | 70.75 |
| purified water | 7.80 |
| sodium monofluorophosphate² | 0.75 |
| preservatives | 0.20 |
| sodium saccharin | 0.10 |
| silica³ | 6.00 |
| silica⁴ | 9.00 |
| thixotropic agent⁵ | 2.00 |
| sodium lauryl sulphate | 1.50 |

¹cellulose gum (Blanose ™ 7MFD ex Aqualon Co.)
²Phoskadent ™ Na 211 ex BK Giulini Chemie, Germany)
³Syloblanc ™ 81 ex Grace, Germany
⁴Syloblanc ™ 82 ex Grace, Germany
⁵Aerosil ™ 200 ex Degussa, Germany

To 98 parts by weight of this formulation, 2 parts of capsules were incorporated. The samples were allowed to equilibrate at room temperature for two weeks. After two weeks, the capsules were removed from the tooth-gel. The tooth-gel was analyzed to determine the amount of flavour that had partitioned from the capsule core. The flavour was extracted from the tooth-gel, utilizing a mixture of 80% hexane and 20% acetone, as the extraction solvent analyzed by GC FID. It was found that the capsules according to the first type had better flavour retention, 22% flavour partitioned to the tooth-gel base, whereas those that contained 100% flavour as the core material had 49% of the flavour partition to the tooth-gel base.

### EXAMPLE 2

Hydrogel capsules were made as described in Example 1. The core of the capsule contained a blend of 10 wt % mint flavour, flavour blend having a calculated Log P_{OCS} of 2.0 and 90 wt % of dilution solvent migylol (MCT Oil). The capsules had a particle size range of 500 to 1000 microns. The flavored capsules were loaded into an unflavored tooth-gel formulation, as described in Example 1, at 2 wt % load.

The formulations were subjected to accelerated aging studies performed for 12 weeks at 40° C., which approximates to two years at ambient conditions (the endurance expected from a tooth-gel).

After 12 weeks, the capsules in the toothgel were evaluated under different criteria:

Sensory Testing—fresh sample vs. 12 week @ 40° C. aged sample evaluating flavour intensity

Capsule integrity—microcapsules still intact visual assessment by microscopy.

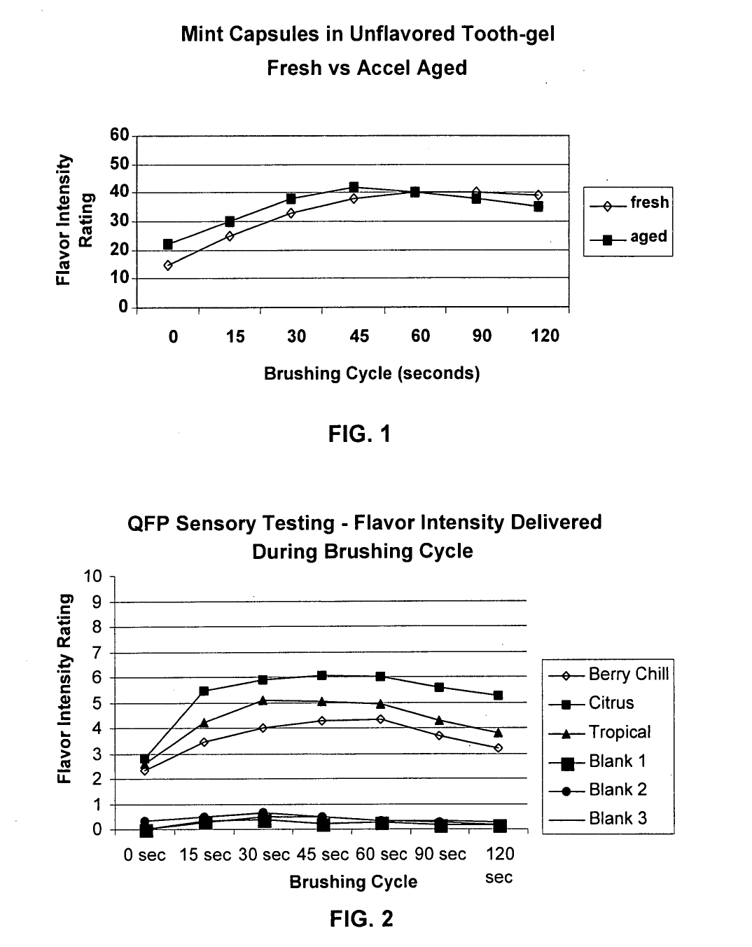

Sensory testing was done by a trained panel. The panel evaluated the samples by the members brushing their teeth with the sample for 120 seconds, and rating the sample for flavour intensity at 15 sec, 30 sec, 45 sec, 60 sec, 90 sec and 120 sec. Sensory testing results showed the aged sample to have a flavour intensity profile during the brushing cycle similar to that of the fresh sample. These results are shown in FIG. 1. Capsule integrity was evaluated by microscopy. Results indicate that the capsules are stable in the tooth-gel product.

### EXAMPLE 3

Hydrogel capsules were made as described in Example 1. In this case, three types of flavour capsules were made. The first contained a blend of 20 wt % berry flavour blend having a calculated Log P_{OCS} of 2.3, and 80 wt % of dilution solvent Migylol (MCT Oil). The second contained a blend of 20 wt % tropical flavour blend having a calculated Log P_{OCS} of 2.6 and 80 wt % of dilution solvent migylol (MCT Oil). The third contained a blend of 20 wt % citrus flavour blend having a calculated Log P_{OCS} of 2.2 and 80 wt % of dilution solvent migylol (MCT Oil). As a control, a hydrogel capsule containing Migylol (MCT Oil) as the core material was made. The capsules had a particle size range of 500 to 1000 microns. The flavored capsules and blank capsules were loaded separately into a 1 wt % mint flavored tooth-gel formulation at 2 wt % load. The formulation was identical to that described in Example 1, with the difference that 1 part of water is replaced by 1 part of a proprietary mint flavour ex Givaudan Flavors Corp. To 97 parts of this formulation was added 2 parts of microcapsules and 1 part of the mint flavour.

The samples were allowed to equilibrate at room temperature for two weeks. After two weeks, the samples were analyzed for flavour intensity during the brushing cycle. Sensory testing was done by a trained panel. The panel evaluated the samples by brushing their teeth with the sample for 120 seconds, and rating the sample for flavour intensity at 15 sec, 30 sec, 45 sec, 60 sec, 90 sec and 120 sec. The panelists were looking for a second flavour profile being released during brushing.

Sensory testing results showed the flavored capsules in tooth-gel released a distinguishable secondary flavour during the brushing cycle. The blank capsules in tooth-gel did not give a secondary flavour profile. FIG. 2 shows the flavour intensity curves for the berry, citrus and tropical flavored capsules. The capsules were rupturing and were delivering a distinguishable secondary flavour during the brushing cycle.

### EXAMPLE 4

Hydrogel capsules were made as described in Example 1. In this case, the capsules were made with ratios of core diameter to wall thickness of 12:1 and 20:1. The core of the capsule contained a blend of 10 wt % mint flavour blend having a calculated Log P_{OCS} of 2.0 and 90 wt % of dilution solvent migylol (MCT Oil). The two ratios were also each made at two particle size ranges, 500 to 1000 microns and 1000 to 2500 microns. The flavored capsules were loaded into an unflavored tooth-gel formulation, as described in Example 1, at 2 wt % load.

The samples were analyzed by Hardness Testing to determine the extent of capsule rupture during the brushing cycle. Hardness Testing was done on an ADA Testing Machine V8 Cross Brushing Machine. The following procedure was used for Hardness Testing:

V-8 Brushing Machine Method:

1. Adjust tension on machine to desired force (grams). Calibrate with spring tensiometer to verify force setting.
2. Weigh out 1.0 grams of tooth-gel product on spatula. Deposit on toothbrush.
3. Wet toothbrush with 5.0 ml of deionized Water.
4. Place plastic container over assembly.
5. Turn on machine brush for 30 seconds. Turn off machine.
6. Remove plastic container from top of assembly. Rinse brush and teeth with 50 ml de-ionized water. Collect in plastic container.
7. Remove plastic container from assembly. Filter slurry through funnel with filter paper (Qualitative 4 Filter Paper). Collect capsules on filter paper.
8. Evaluate capsules on filter paper under microscope at 4× magnification. Count number of intact capsules and number of ruptured capsules.
9. % Capsule Rupture=Broken Capsules in sample/Total Capsules in sample*100

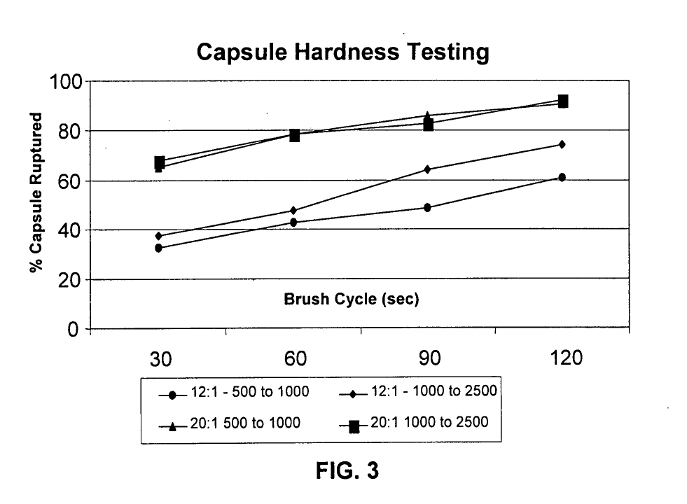

Hardness testing results are depicted in FIG. 3. They show that capsule wall thickness affects how the capsules rupture during the brushing cycle. Capsules with a core to wall ratio of 12:1 had a capsule rupture rate of 60% to 70% after brushing for 120 seconds, while capsules with a core to wall ratio of 20:1 had a capsule rupture rate of 90% to 92% after brushing for 120 seconds. The thinner the wall of the capsule, the higher the capsule rupture rate during brushing.

### EXAMPLE 5

Hydrogel capsules were made as described in Example 1. In this case, a concentration series was run with Peppermint Oil and Migylol as the core materials, the capsule contents being as follows:
Capsule A—10% peppermint oil, 90% miglyol
Capsule B—20% peppermint oil, 80% miglyol
Capsule C—50% peppermint oil, 50% miglyol
Capsule D—100% peppermint oil

The capsules had a particle size range of 500 to 1000 microns. The flavored capsules were loaded separately into an unflavored tooth-gel formulation at 2 wt % load. The formulation was identical to that described in Example 1.

The samples were allowed to equilibrate at room temperature for two weeks. After two weeks, the capsules were removed from the tooth-gel. The tooth-gel was analyzed to determine the amount of flavour that had partitioned from the capsule core. The flavour was extracted from the tooth-gel, utilizing a mixture of 80% hexane and 20% acetone, as the extraction solvent analyzed by GC FID. It was found that the capsules containing the active and the solvent as defined had better flavour retention. The weight percentages of peppermint flavouring lost to the tooth-gel formulation were as follows:
Capsule A—30%
Capsule B—45%
Capsule C—65%
Capsule D—95%

While the microcapsules, compositions incorporating microcapsules and methods have been described above in connection with certain illustrative embodiments, it is to be understood that other similar embodiments may be used or modifications and additions may be made to the described embodiments for performing the same function(s). Further, all embodiments disclosed are not necessarily in the alternative, as various embodiments may be combined to provide the desired characteristics. Variations can be made by one having ordinary skill in the art without departing from the spirit and scope of the disclosure. Therefore, the microcapsules, compositions containing the microcapsules, and methods should not be limited to any single embodiment, but rather construed in breadth and scope in accordance with the recitation of the attached claims.

We claim:

1. Microcapsules comprising hydrogel walls and a content comprising a flavour or fragrance active and a solvent therefor, the solvent having a Clog P>5, the solvent being present in such a proportion that the active in solution has a calculated base-ten logarithm of the partition coefficient between the solvent and a continuous aqueous phase containing 1.5% by weight anionic surfactant (Log P_{OCS}) of at least 1.7.

2. The microcapsules according to claim 1, wherein the Log P_{OCS} is at least 2.

3. The microcapsules according to claim 1, in which the ratio of the diameter of the capsule to the capsule wall thickness, R, is at least about 10:1

4. The microcapsules according to claim 2, in which the ratio of the diameter of the capsule to the capsule wall thickness, R, is at least about 12:1.

5. The microcapsules according to claim 2, in which the ratio of the diameter of the capsule to the capsule wall thickness, R, is at least about 16:1.

6. The microcapsules according to claim 2, in which the ratio of the diameter of the capsule to the capsule wall thickness, R, is at least about 20:1.

7. The microcapsules according to claim 1, in which the capsule diameter is between about 100 and about 2500 micrometres.

8. The microcapsules according to claim 1, in which the capsule diameter is between about 100 and about 2000 micrometres.

9. A composition in which encapsulation of flavour or fragrance is desired, comprising ingredients appropriate to said composition and the microcapsules according to claim 1.

10. The composition according to claim 9, in which one of said ingredients is surfactant, present in the composition at a content of from about 1% to about 10% by weight.

11. The composition according to claim 10, in which the composition is at least one of toothpaste, tooth-gel, laundry detergent, fabric softener, hair care product, cosmetic cream, medicinal cream, or personal cleansing product.

12. The composition according to claim 11 wherein the hair care product comprises a shampoo or a conditioner.

13. The composition according to claim 11 wherein the personal cleansing product comprises a shower gel, a body lotion, or a soap.

14. A method of increasing the storage life of encapsulated flavour or fragrance active in a composition that comprises a high surfactant environment, comprising incorporating the active in the microcapsules according to claim 1 and blending the microcapsules into the composition.

15. A method of increasing the storage life of encapsulated flavour or fragrance active in a composition that comprises at least about 1% surfactant, comprising incorporating the active in the microcapsules according to claim 1 and blending the microcapsules into the composition.

16. The method according to claim 15 wherein the composition comprises surfactant, at a content of from about 1% to about 10% by weight of the composition.

17. The method according to claim 15, wherein the Log P_{OCS} is at least 2.

18. The method according to claim 15, in which the ratio of the diameter of the capsule to the capsule wall thickness, R, is at least about 10:1.

19. The composition according to claim 9, wherein the Log Pocs is at least 2.

20. The composition according to claim 9, in which the ratio of the diameter of the capsule to the capsule wall thickness, R, is at least about 10:1.
